(19) United States
(12) Patent Application Publication
(10) Pub. No.: US 2022/0141448 A1
(43) Pub. Date: May 5, 2022

(54) Method for Operating a 3D Body Scanner

(71) Applicant: Naked Labs Austria GmbH, Wien (AT)

(72) Inventors: Gerhard Schultes, Wien (AT); Farhad Farahbakhshian, Redwood City, CA (US); Peter Kreuzgruber, Wien (AT)

(21) Appl. No.: 17/434,121

(22) PCT Filed: Feb. 21, 2020
(86) PCT No.: PCT/EP2020/054699

§ 371 Date: Aug. 26, 2021

(30) Foreign Application Priority Data
Feb. 27, 2019 (AT) A 60051/2019

(51) Int. Cl. H04N 13/296; H04N 13/271; H04N 13/257; H04N 5/225

(52) U.S. Cl. CPC H04N 13/296; H04N 13/271; A61B 5/0064; H04N 5/2256; H04N 13/257

(57) ABSTRACT

A method for operating a 3D body scanner, which generates a 3D body model, includes the steps of measuring an installation geometry of the 3D body scanner, calculating an optimal scanning distance between a person to be scanned and the 3D body scanner by a control unit on the basis of at least the geometry of the installation of the 3D body scanner and projecting an optical indicator for the person to be scanned on a floor in front of the 3D body scanner at the optimal scanning distance from the 3D body scanner by a position indicator projector. A 3D body scanner is configured for performing the method for operating the 3D body scanner for generating a 3D body model.

### CROSS REFERENCE TO RELATED APPLICATIONS

The present application claims priority to application Serial No. PCT/EP2020/054699 filed Feb. 21, 2020, which is hereby incorporated herein in its entirety by this reference for all purposes.

### FIELD OF THE INVENTION

The invention relates to a method for operating a 3D body scanner for generating a 3D body model wherein the 3D body scanner includes a control unit and a position indicator projector for projecting an optical indicator for the person to be scanned at a floor in front of the 3D body scanner.

### BACKGROUND OF THE INVENTION

Today accurate 3D human body models are demanded by various fields of applications as are: Fitness and body styling application, Medical applications, Cloth manufacturing industry, Cloth internet- and retail shops and/or Automotive industry.

To generate 3D body models 3D body scanners are used. For high performance scans the human body must be placed as close as possible to the scanner but not so close that it is exceeding its Field of View (FOV).

If the user is too close to the scanner, parts of its body will be cut-off at the top, bottom and/or left or right. If a user is too far it will typically result in a low-resolution scan. State of the art 3D body scanners are well defined installed by standing on a flat floor.

Home scanners are scanning while standing on a turntable mechanically separated from a scanner mast. The turntable is placed manually on the floor in front of the scanner. The concept works well because installation is very easy and there is only a low chance to set an improper distance.

Turntable-less 3D body scanners let the person by the person's own motion turn around at a roughly defined speed.

Common disadvantage of the solutions above is that they need a fix and inflexible construction determining the height and angle of view of the depth sensors installed in the 3D body scanners. This is leading to:

(i) Large scanner constructions which need to be placed on the floor or;
(ii) When considering small, more handy scanners, the need to be installed under fulfilling strict geometrical conditions, which should also not be changed uncontrollably at operation. The required skills and tools can hardly be demanded from an average user. Also changing the operating conditions can hardly be avoided in a normal living space.

### OBJECTS AND SUMMARY OF THE INVENTION

The disadvantages will be solved by a method for operating a 3D body scanner and a 3D body scanner according to the description that follows.

It is proposed a method operating a 3D body scanner for generating a 3D body model. The method of the disclosed invention automatically creates a visible indication in optimum distance on the floor in front of the scanner where the user should step on for scanning. The distance is automatically adapted depending on how the scanner is installed in a room.

At a first step an installation geometry of the 3D body scanner will be measured. Installation geometry means, for example, a position and/or orientation of the 3D body scanner according to a room and/or a floor.

At a further step, a control unit will calculate an optimal scanning distance between a person and the 3D body scanner. The distance must not be too great, otherwise the resolution of the scanner will be degraded. On the other hand, the person must not get too close to the scanner, otherwise the person cannot be fully captured. In general, the optimal scanning distance is as close as possible to the 3D body scanner. Therefore, the optimal scanning distance should be as short as possible. Furthermore, the optimal scanning distance indicates the optimal scanning position in front of the 3D body scanner. The optimal scanning position may be furthermore in the middle in front of the 3D body scanner.

At a further step, an optical indicator for the person to be scanned will be projected on a floor in front of the 3D body scanner at the optimal scanning distance from the 3D body scanner by a position indicator projector. The person to be scanned can move to the optical indicator so that the 3D body scanner can capture the person as well as possible. Therefore, the 3D body scanner can be arbitrarily placed in the room. For example, it does not matter, whether the 3D body scanner is placed on a high or a low furniture.

At a further advantageous step, for the installation geometry a height of the 3D body scanner above the floor in front of the 3D body scanner will be measured.

At a further advantageous step, for the installation geometry an angle of the 3D body scanner 104 according to a vertical axis will be measured.

At a further advantageous step, for calculating of the optimal scanning distance a field of view (FOV) of a depth sensor of the 3D body scanner 104 is taken into account.

At a further advantageous step, for calculating of the optimal scanning distance a field of view (FOV) of a color camera of the 3D body scanner 104 is taken into account. The color camera may be a RGB camera.

At a further advantageous step, the field of view (FOV) will be scanned by the depth sensor and/or by the color camera. At a further step, a depth map will be generated by the control unit on basis of the data of the depth sensor and/or of the color camera. The depth map may contain distance information for each point (according to the resolution of the depth sensor and/or the color camera) of the area of the field of view. The field of view may be 2-dimensional. The field of view may have a width and a height. The field of view further has a vertical and a horizontal expansion. For example, the body height of the person to be scanned can be determined from the depth map.

At a further advantageous step, the floor in front of the 3D body scanner on basis of the depth map will be identified. In general, the floor is a flat plane. This can be analyzed or recognized to identify the floor.

At a further advantageous step, a body height of the person to be scanned will be determined. For example, the control unit can determine the body height on basis of data from the depth sensor and/or the color camera. Furthermore, the body height can be determined on basis of the depth map. In addition or alternatively the body height can be assumed. For example, a body height of 1.80 meter, 1.90 meter or 2 meters can be assumed.

At a further advantageous step, the height of the 3D body scanner above the floor and/or the angle of the 3D body scanner to the vertical axis on basis of the depth map will be calculated.

At a further advantageous step, the optical indicator will be guided by controlling the position indicator projector. The position indicator projector is advantageously connected to the control unit. The control unit can control the position indicator projector in such a way that the position indicator projector will project the optical indicator at the optimal scanning position, which is arranged at the optimal scanning distance from the 3D body scanner and in particular in the middle in front of the 3D body scanner.

At a further advantageous step, the person to be scanned will be guided to the optical indicator by optical and/or audible signs. The user or the person to be scanned will be guided to the optimum position in front of the scanner for scanning and this position is automatic adapted to the body height of the user and how the scanner is installed in a room.

For guiding the user or person to be scanned, the 3D body scanner may comprise a direct communication interface, in particular a speaker, a buzzer, LED indicators as, for example, arrows and/or a display. Signs to guide the person to be scanned to the optimal scanning position can also be projected by the position indicator projector.

The method for interactive guiding the person to the optimum scanning position follows according at least one of the following steps. The user approaches the FOV of the 3D body scanner and the 3D body scanner switches, either user initiated or automatically from standby to active scanning mode. Then 3D body scanner determines the actual position of the user and starts guiding the user interactively by directions/advices to the optimum scanning position. The optimum scanning position is arranged at the optimum scanning distance, in particular furthermore in the middle, in front of the 3D body scanner. The advantage of this solution is that, by intuitively accepting to follow the directions/advices of the 3D body scanner to change the position of the body, no special user action is required. The interactive guiding of the user to the optimum scanning position is the following iterative process:

The 3D body scanner determines the geometrical relations (for example, but not limited to, distances, angles of floor, walls, objects, obstacles, user) in front of it and/or the geometrical installation of the 3D body scanner by its sensing components. This means 3D body scanner records, for example, depth map(s), which is in principle a 2D matrix of distance values, with its depth sensor(s) and/or RGB or black-white image(s) by its camera(s) of the room in front of it and optionally, if the user is already sufficiently visible, the body height of the user. Additionally or alternatively, the 3D body scanner may measure location data of, for example, but not limited to, objects or persons by Lidar or microwave radar.

The 3D body scanner calculates by geometrical equations from the geometrical relations and/or location data, the optimum scanning position of the user on the floor. This is done, for example, but not limited to, analyzing recorded depth maps, black-white, RGB or IR images and/or analyzing the location data.

The 3D body scanner determines the actual position of the user on the floor. This is done, for example, but not limited to, analyzing recorded depth maps, black-white, RGB or IR images and/or analyzing the location data.

The 3D body scanner calculates the length and direction of a difference vector between the actual position of the user and the optimum scanning position on the floor. This is done, for example, by its computation unit or within the computation unit of external device(s) or by cloud based processing.

The 3D body scanner checks if the actual position of the user is within a given circle of sufficient accuracy close to the optimum scanning position. If yes, the guiding process is ended.

If no, the 3D body scanner gives the user in an advantageous way a direction/advice how to move to the optimum scanning position. The direction/advice to the user may contain in advantageous way, for example, but not limited to, directional and/or distance information in absolute or relative scale. The direction/advice can be given, for example, either fix related to the main axis of view of the 3D body scanner or adaptive related to the direction of view of the user. The directions/advices to the user can be transmitted in an advantageous way, directly by the direct communication interfaces or indirectly by the indirect communication interfaces via connected external device(s).

The direction(s)/advice(s) given to the user may be of acoustical-, or optical or any other human noticeable nature. This can be for an acoustic way, in particular verbal (for example, voice) or non-verbal (for example, sound) and/or an optical way as, for example, written (static, running or animated text), static symbols or icons (for example, arrows or pictograms) and/or dynamic (live) symbols, icons or animations.

The 3D body scanner gives the user some time to move.

If the person is at the optimal scanning position the 3D body scanner gives a message that it is ready to scan or a given maximum number of iterations is reached and the 3D body scanner gives the message that a scan is not possible.

External devices can be, for example, laptops, smartphones, tablets, and/or intelligent personal assistants.

Furthermore, a 3D body scanner for generating a 3D body model is proposed. The 3D body scanner is designed in such a way that the 3D body scanner can perform the method described in the previous and/or the following description. For example, the 3D body scanner may comprise a control unit, a position indicator projector and a sensor, in particular a depth sensor and/or a color camera, to measure the installation geometry of the 3D body scanner. The sensor, in particular a depth sensor and/or a color camera, can additionally or alternatively be used to scan the field of view to determine the body model of the user or the person.

In an embodiment of the 3D body scanner, the 3D body scanner and/or the position indicator projector comprises an actuator for tilting the position indicator projector, for tilting a mirror of the position indicator projector to deflect a light and/or laser ray and/or for adjusting a lens of the position indicator projector. With at least one of these features, the optical indicator can be guided to the optimal scanning distance.

In a further embodiment of the 3D body scanner, the position indicator projector comprises at least a light source, in particular LEDs, lasers, tungsten or halogen lamps. With at least one of these features, the optical indicator can be projected on the floor. Position indicator projector is designed in such a way that the position indicator projector can project, for example, a circle, a square and/or a star at the floor to indicate the optimal scanning distance for the person to be scanned. Furthermore, the position indicator projector is designed in such a way that the position indicator projector can project, for example, an arrow to show the person to be scanned the way to the optimal scanning distance or the optimal scanning position in front of the 3D body scanner.

In a further embodiment of the 3D body scanner, the 3D body scanner comprises an accelerometer for measuring the height of the 3D body scanner and/or an angle of the 3D body scanner according to a vertical axis.

The depth sensor is realized, for example, on basis of Stereo vision, assisted stereo vision, structured light, Time of flight, Lidar and/or Radar.

The optical and/or color camera is realized, for example, on basis of RGB, black-white and/or IR.

Additional advantages of the invention are described in the following exemplary embodiments.

### BRIEF DESCRIPTION OF THE DRAWINGS OF EXEMPLARY EMBODIMENTS

The drawings show in:

FIG. 1 two 3D body scanners at different heights,

FIG. 2 position indicator projector with tilted light or laser ray,

FIG. 3 position indicator projector with active light or laser ray and

FIG. 4 top view on the floor with the 3D body scanner, the user or person and the optimal scanning position.

### DETAILED DESCRIPTION OF EXEMPLARY EMBODIMENTS

The 3D body scanner can be in a small size with the intention of facilitating placement of the 3D body scanner somewhere in a desired environment, which includes a floor, whether on a piece of furniture or hung on a wall in an only very roughly defined height and/or angular orientation. Usually, but not necessarily, turntables will be omitted. Nevertheless, for a best performance body scan, the user still needs to fill, as far as possible, but not exceed the field of view (FOV) in front of the 3D body scanner. The large amount of freedom of placement in height and angular orientation of installation on furniture creates a situation dependent on compromising of the scanning distance of the user. This means that the optimum scanning distance will change with the parameters of installation. The proposed inventive method will adaptively project an optical position indicator on the floor in front of the 3D body scanner in a distance which is optimum with respect to an installation geometry, in particular to installation height and placement angle of the 3D body scanner, for a given of a maximum body height of the user. This process is advantageously automatically run prior to any scan and works typically in three steps: (1) Determination of optimum scanning distance by measuring of installation geometry and geometric calculations; (2) Open- or closed-loop movement of light or laser ray angle using an electro-mechanical or electronic actuator; (3) Projecting of a convenient position indicator on the floor.

For simplicity of explanation in the next sections we state that the 3D body scanner emits a single light ray or laser ray and the optical indicator is assumed to be a single point at the optimum scanning position, even if it is a circle, ellipse, cross, star or any other convenient figure or pattern. Nevertheless, the 3D body scanner can also emit multiple light rays or laser rays.

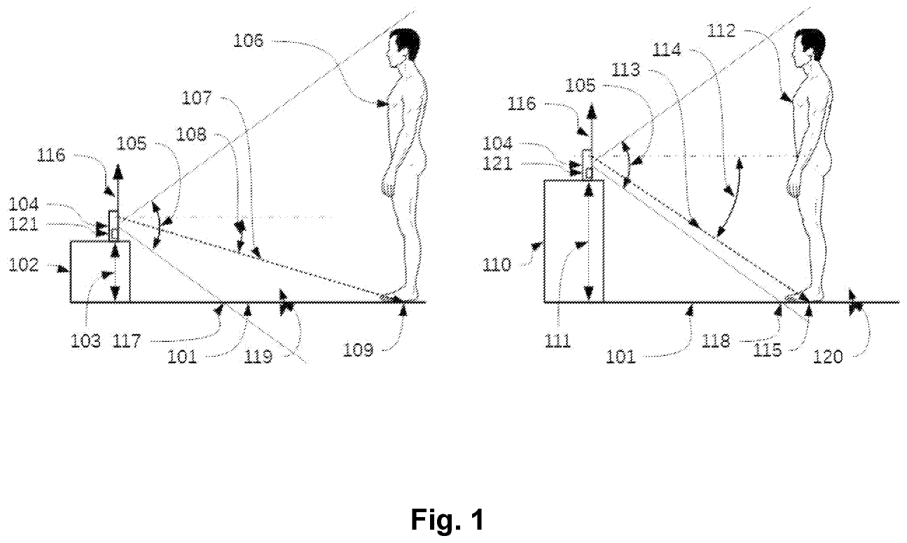

According to FIG. 1, two configurations of 3D body scanners 104 are shown deployed at different heights 103, 111. The optimum scanning distances are different when the 3D body scanners 104 are placed at different heights 103, 111 and the height axis 116 of 3D body scanner 104 is not fully vertical. The optimum scanning distances will be determined as given in the following examples:

The 3D body scanner 104 is placed, for example, on a low furniture 102 with the FOV 105 of the 3D body scanner 104 looking in the room. This configuration is shown on the left side of FIG. 1. The person 106 to be scanned at this low placed scanner 104 needs to be in a comparable far position. A limiting factor of the placement of the person 106 is that the person's head is still in the FOV 105 of the 3D body scanner 104. To determine the optimum scanning position of the person to be scanned 106, 3D body scanner 104 can do the following steps in accordance with the present invention:

The 3D body scanner 104 records a depth map of the room in front of the 3D body scanner 104 and identifies the floor 101 as a plane in front of the 3D body scanner.

The starting distance 117 of the floor plane 101 is determined in the FOV 105 of the low placed scanner 104.

The viewed angle 119 of the floor plane 101 is related to vertical height axis 116 of the 3D body scanner. In an alternative embodiment when the 3D body scanner has a built-in accelerometer, then the viewed angle 119 of the floor plane 101 is related to the angle of the vertical height axis 116 of the 3D body scanner 104.

There is a-priori knowledge of the FOV 105 of the 3D body scanner 104.

An assumption of the maximum height 112 of the person 106 to be scanned is made in accordance with the method of the invention.

Therefrom, the 3D body scanner 104 calculates by geometrical relations the optimum scanning distance for the optical indicator 109 on the floor 101 for the low placed scanner 104. When turning on the light or laser ray 107 of the low placed scanner 104, an optical indicator 109 which shows where the user 116 has to step on, is projected on the floor 101.

Furthermore, the 3D body scanner 104 can also be placed, for example, at a higher elevation on a piece of furniture 110 with its FOV 105 looking into the room. This configuration is shown on the right side of FIG. 1. The person 112 to be scanned by the high placed scanner 104 can be positioned in a comparatively close position to the 3D body scanner 104. This relative placement increases the resolution of the scanned person 112. A limiting factor on this placement is in this case that the feet of the person 112 must still be within the FOV 105 of the high placed 3D body scanner 104. To determine optimum scanning position of the person 112 to be scanned, the 3D body scanner 104 calculates the angle 114 of light or laser ray to project an optical indicator 115 on the floor 101.

If the installation of the 3D body scanner 104 exceeds given limits and no appropriate scanning distance can be found, then the method of operating the 3D body scanner 104 may give in an advantageous way notice to the user 106, 112. The notice may be, but not limited to, a notice that is an optical- or acoustical-, wired- or wireless-electronic notice.

A feature of this invention is that the optical indicator can be moved to adapt according to the scanner installation geometry. There are at least, but not limited to, two advantageous ways to perform this movement: (1) Electro-mechanical movement and/or (2) Electronic movement.

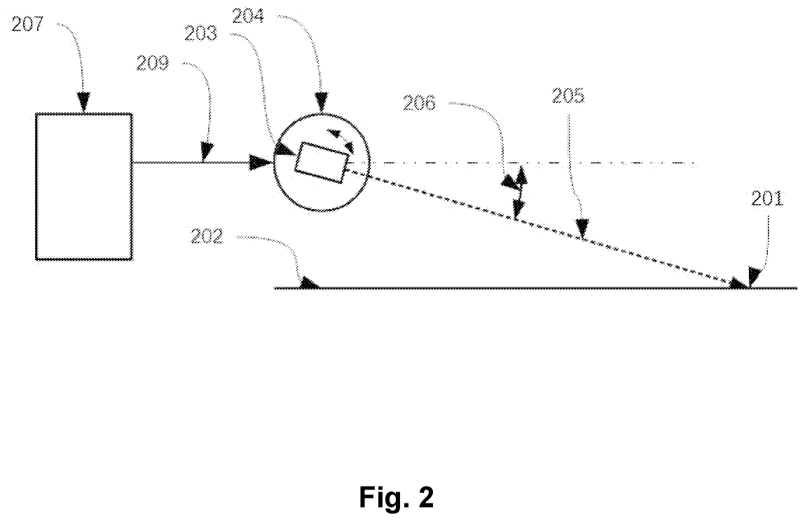

According to FIG. 2, the position indicator projector 203 is schematically shown.

Features which are already described in the previous figure might not be explained again for the sake of simplicity. Furthermore, features may only be described in subsequent figures. Furthermore, for the sake of clarity, not all characteristics may be shown in the following figures. However, characteristics shown in one or more of the preceding figures may also be present in one or more of the following figures. Furthermore, for the sake of clarity, characteristics shown in one or more of the following figures may also be shown in one or more of the following figures. Nevertheless, features that are shown in one or more of the following figures may also be present in a previous figure. Furthermore, different reference signs can be used for the same features in the FIGS. 1-4.

In the electro-mechanical solution, the control unit 207 of the 3D body scanner provides the angular position information as a digital or analog control signal 209 to an electro-mechanical actuator 204. This signal 209 causes the actuator 204 to change the angle of the position indicator projector 203 and therewith the angle 206 of the light or laser ray 205. The light or the laser ray 205 is projected onto the floor 202. The electro-mechanical actuator 204 affects the motion of the ray 205 of the position indicator projector 203:

Some advantageous analog electro-mechanical implementations can include, but not limited to, a rotatable or shiftable galvanometer coil, a piezo-electric actuator, or a servo motor.

Some advantageous digital implementations can include, but not limited to, a rotating or linear stepper motor.

In any other electro-mechanical way, which allows changing the position of the optical indicator 109, 115, 201 on the floor 101, the optical indicator 201 projected on the floor 202 may have in an advantageous way any shape which is best visible or otherwise convenient to the user. It may be, for example, but not limited to, a simple dot, a cross-hair, star, a filled or hollow square, circle or ellipse. Complicated indicator figures can be realized in an advantageous way by:
using a beam shaped laser and/or
using a lens-based position indicator projector 203

An also advantageous alternative electro-mechanical solution is using a fix mounted projector and a movable mirror tilted by an electro-mechanical actuator for changing the direction of the light or laser beam 205. This movable mirror can be a conventional mechanical solution, as, for example, mentioned above, or a solid-state circuit MEMS.

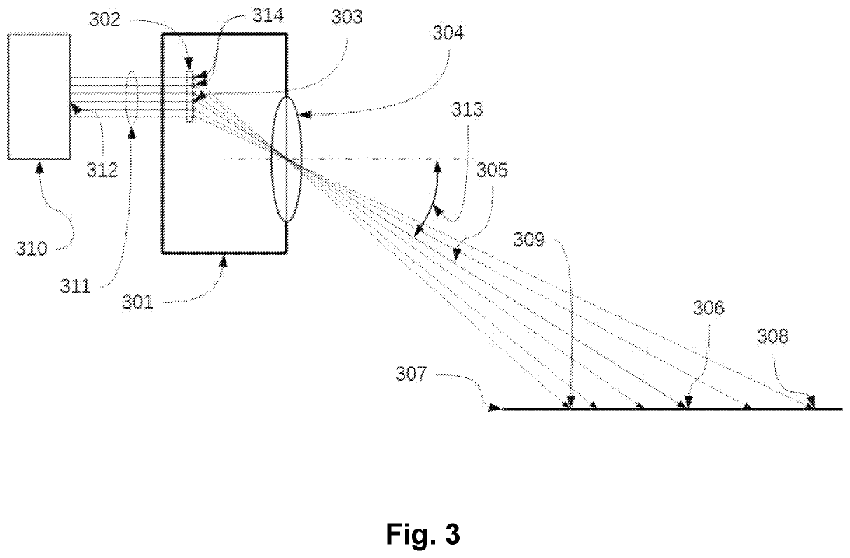

FIG. 3 schematically shows a further position indicator projector 301.

Features which are already described in one or more of the previous figures may not be explained again for the sake of simplicity. Furthermore, features may only be described in subsequent figures. Furthermore, for the sake of clarity, not all characteristics may be shown in the following figures. However, characteristics shown in one or more of the preceding figures may also be present in one or more of the following figures. Furthermore, for the sake of clarity, characteristics shown in one or more of the following figures may also be shown in one or more of the following figures. Nevertheless, features that are shown in one or more of the following figures may also be present in a previous figure. Furthermore, different reference signs can be used for the same features in the FIGS. 1-4.

The invented electronic concept is based on projecting in an advantageous simple way, the image of a light source 303, which is a part of an electronic actuator 302, by a projection lens 304 on the floor 307. This electronic concept is advantageous because it is noiseless and avoids costly and error-prone mechanical moving parts. The control unit 310 of the 3D body scanner 104 provides the angular position information 311 as one or more control signal(s) 312 to an electronic actuator 302, which is a vector (line) that includes a plurality of switchable light sources 314.

To change the tilt angle 313 of the projected light or laser ray 305, and therewith the position of the light indicator 306 on the floor 307, only one or a limited number of such light sources 314 is activated by the control signal(s) 312 from a control unit 310. Dependent on the position of the active light source(s) 303 on the electronic actuator 302, the light or laser ray 305 can be moved between the closest light indicator position 309 on the floor 307 and the farthest light indicator position 308 on the floor.

The granularity (distance steps) of the different discrete positions can be in an advantageous way selected by the number and distance of light sources in the vector (line) of light sources 302 and the angle of the electronic actuator 302 with respect to the optical axes of the projection lens 304. The light sources 314 may be in an advantageous way of any physical kind, such as but not limited to, LEDs, lasers, tungsten or halogen lamps, or whatever light sources that can be manufactured in suitable format. The optical indicator 306 projected on the floor 307 may have in an advantageous way any shape which is best visible or otherwise convenient to the user. It may be, for example, but not limited to, a simple dot, a cross-hair, star, a filled or hollow square, circle or ellipse. Complicated static or dynamic symbols or figures can be realized in an advantageous way by:
Composing the figures by multiple dots whereas the electronic actuator 302 can become a two-dimensional matrix of light sources or,
Using for the actuator 302 a vector of beam shaped lasers.
Using an LCD monitor instead of a simple vector or matrix of light sources, which selectively allows the projection of a stand still or dynamic “alive” images.
Rotating the shape of the indicator could give to the user in an advantageous way a hint about the targeted rotating speed at scanning.

The movement of the optical indicator to the appropriate scanning distance can be done, at least but not limited to, in two ways:

1. Open loop setting (steering) to save in an advantageous way hardware and software effort where the angle of light or laser ray 108, 114 for the targeted indicator position 109, 115 is simply calculated by the control unit 207, 310 of the scanner 104 and set without feedback by the electromechanical-actuator 204 or electronic-actuator 302.
2. Optional closed loop control to increase in an advantageous way accuracy and to combat manufacturing tolerances, where the calculated distance (position) of the optical indicator on the floor 109, 115 is set as a target, and the real position of the indicator is moved by the electro-mechanical-204 or electronic-302 actuator in a closed loop using the difference between the target position and the actual indicator position from a GB image captured by an optional RGB camera 121 of the scanner 104 as feedback information.

It is also advantageous that the concept can be used together with a turntable. In this case, the height of the turntable has to be provided to the control unit of the 3D body scanner. Then, this height information can correct its calculation by this height and project the indicator position for the optimum location of the turntable. It is also possible that the scanner itself identifies the turntable and/or its height and automatically performs this correction. This identification can be done from a depth map image and/or RGB image.

The invention is not limited to the embodiments shown or described. Rather, any and all combinations of the individual features described, as shown in the figures or described in the description, and to the extent that a corresponding combination appears possible and sensible, are subject matters of the invention.

An advantageous aspect is the capability to adaptively project an optical position indicator 109, 115 on the floor 101 in front of the 3D body scanner 104 in a distance which is in an advantageous way optimum with respect to installation height 103, 111 and placement angle 116 of the 3D body scanner 104 for a given of a maximum body height of the user 106,112.

An advantageous aspect is that the determination of the optimum distance is performed prior to every scan to ensure that intentional changes or changes by chance in the installation geometry of the 3D body scanner 104 are considered.

An advantageous aspect is that the 3D body scanner 104 gives to the user, notice which may be, but not limited to, in optical- or acoustical-way, or a wired- or wireless-electronic way, if the geometrical relations of the installation are out of limit and/or no appropriate scanning distance can be found.

An advantageous aspect is [[that]] an electro-mechanical concept of optical indicator movement. As schematically shown in FIG. 2, a control unit 207 provides an angular position information as a digital or analog control signal 209 to an electro-mechanical actuator 204 which can change the angle of the indicator projector 203 and consequently the angle 206 of the emitted light or laser ray 205 or directly angle 206 of the emitted light or laser ray 205 to change the distance of the optical position indicator 201.

An advantageous aspect is that the electro-mechanical actuator 204 is, in an advantageous analog electro-mechanical way, a, but not limited to, rotatable or shiftable galvanometer coil, a piezo-electric actuator, or a servo motor or in an advantageous digital way a, but not limited to, rotating or linear stepper motor, or any other electro-mechanical way.

An advantageous aspect is that the electro-mechanical solution with a fixed mounted projector and a tilt-able mirror, is moved by an electro-mechanical actuator for optical indicator distance control.

An advantageous aspect is schematically shown in FIG. 3, wherein the electronic concept of optical indicator movement is based on projecting in an advantageous way the image of a light source 303 by a projection lens 304 on the floor 307, and to change the angle 313 of the projected light or laser ray 305 in a manner that is noiseless, without any mechanically moving parts, by using an electronic actuator 302, which is vector (line) of consecutive light sources 314, activated by control signal(s) 312, whereas only one, or a limited number of the light sources 314, is made actively illuminating 303 and dependent on the position of the active light source(s) 303, the projected optical position indicator 306 is moved in distance on the floor 307.

An advantageous aspect is that the optical indicator projected on the floor may have in an advantageous way any shape, which is best visible or otherwise convenient to the user as is, for example, but not limited to, a simple dot, a cross-hair, a filled or hollow circle or ellipse.

An advantageous aspect is that the optical indicator projected on the floor 307 may be in an advantageous way a standstill, alive or rotating image, which is electronically shiftable, displayed by an LCD screen, which is acting as an electronic actuator 302.

An advantageous aspect is that the light sources 314 may be in an advantageous way of any physical kind, as but not limited to, LED's, lasers tungsten or halogen lamps, or whatever light sources that can be manufactured in suitable format.

An advantageous aspect is that to save hardware and software effort, the present invention is able to use open loop setting (steering), where in an advantageous way, the angle 105, 206, 313 of light or laser ray 107, 205, 305 for the targeted indicator position is simply calculated by the control unit 207, 310 of the scanner and set without feedback by the electro-mechanical-204 or electronic-actuator 302.

An advantageous aspect is that to increase, in an advantageous way, the accuracy of positioning and to combat manufacturing tolerances by using closed loop control, where the calculated distance (position) of optical indicator 109, 115, 201, 306 on the floor 101, 202, 307 is set as target and the real position of the indicator is moved by the electro-mechanical-204 or electronic-302 actuator and a closed loop using the difference between the target and actual indicator position from a RGB image captured by an optional RGB camera 121 of the scanner 104 as feedback information for the control unit 207, 310 of the scanner 104.

Next generation of 3D body scanners will be configured in a small size with the intention to be placed somewhere on a piece of furniture or hung on a wall in an only very roughly defined height and angular orientation. For a best performance a body scan, the user needs to fill, but not exceed the Field Of View (FOV) in front of the 3D body scanner, whereas the optimum scanning distance will change with the parameters of installation. In accordance with the present invention, a method is provided to project an optical position indicator on the floor in front of the 3D body scanner in a distance which is optimum with respect to installation height and placement angle of the 3D body scanner for a given maximum body height of the user.

As can be seen, the features of the different figures can have different reference signs. For example, the floor has the reference sign 101 in FIG. 1, the reference sign 202 in FIG. 2 and the reference sign 307 in FIG. 3. Nevertheless, the feature may be the same. The same may hold for the other features.

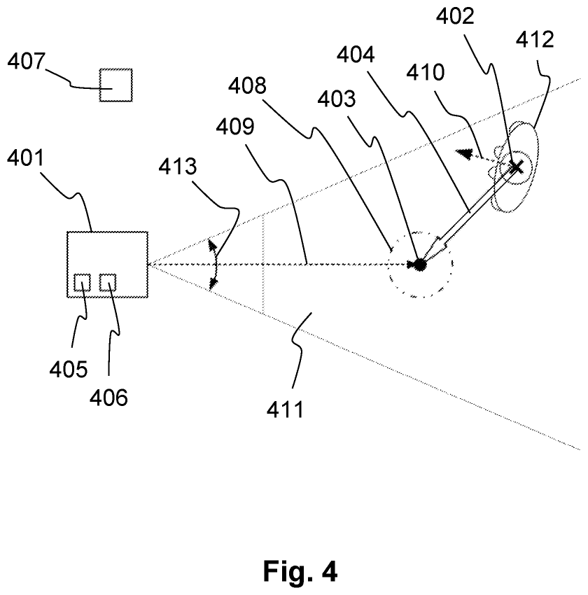

FIG. 4 shows a top view of the 3D body scanner looking down toward the floor 411, the user or person 412 and the optimal scanning position 403. Features which are already described in one or more of the previous figures may not be explained again for the sake of simplicity. Furthermore, features may only be described in subsequent figures. Furthermore, for the sake of clarity, not all characteristics may be shown in the following figures. However, characteristics shown in one or more of the preceding figures may also be present in one or more of the following figures. Furthermore, for the sake of clarity, characteristics shown in one or more of the following figures may also be shown in one or more of the following figures. Nevertheless, features that are shown in one or more of the following figures may also be present in a previous figure. Furthermore, different reference signs can be used for the same features in the FIGS. 1-4.

The user approaches the field of view 413 of the 3D body scanner 401, and in accordance with the operating method of the present invention the 3D body scanner 401 switches, either user initiated or automatically from standby, to active scanning mode. Then the 3D body scanner 401 determines the actual position 402 of the user 412 and starts guiding the user 412 interactively by directions/advices to the optimum scanning position 403. The advantage here is that, by intuitively accepting to follow the directions/advices of the 3D body scanner 401 to change the position of the body, no special user action is required. The interactive guiding of the user to the optimum scanning position 403 is the following, in particular iterative, process:

The 3D body scanner 401 determines the geometrical relations (for example, the distances, angles of floor, walls, objects, obstacles, user) in front of the 3D body scanner by its sensing components. This means, the 3D body scanner 401 records, for example, depth map(s), which is in principle a 2D matrix of distance values, with its depth sensor(s) and/or RGB or black-white image(s) by its camera(s) of the room in front of it and optionally, if the user is already sufficiently visible, the body height of the user. Additionally or alternatively, the 3D body scanner 401 may measure location data of, for example, but not limited to, objects or persons by Lidar or microwave radar.

The 3D body scanner 401 calculates by geometrical equations from the geometrical relations and/or location data, the optimum scanning position 403 of the user on the floor 411. This is done, for example, but not limited to, analyzing recorded depth maps, black-white, RGB or IR images and/or analyzing the location data.

The 3D body scanner 401 determines the actual position of the user 402 on the floor 411. This is done, for example, but not limited to, analyzing recorded depth maps, black-white, RGB or IR images and/or analyzing the location data.

The 3D body scanner 401 calculates the length and direction of the difference vector 404 between the actual position 402 of the user 412 and the optimum scanning position 403 on the floor 411.

This is done, for example, by a computation unit of the 3D body scanner or within the computation unit of external device(s) 407 or by cloud based processing. The computation can also be done by the control unit 310 schematically shown in FIG. 3.

The 3D body scanner 401 checks if the actual position 402 of the user 412 is within a given circle of sufficient accuracy 408 close to the optimum scanning position 403. If yes, then the guiding process is ended.

If no, then the 3D body scanner 401 gives the user in an advantageous way a simple direction/advice how to move to the optimum scanning position 403. The direction/advice to the user 412 may contain in advantageous way, for example, but not limited to, directional and/or distance information in absolute or relative scale. As schematically shown in FIG. 4, the direction/advice can be given, for example, either directionally related to the main axis 409 of view of the 3D body scanner 401 or adaptively related to the direction of view 410 of the user 412. The directions/advices to the user 412 can be transmitted in an advantageous way, directly by the direct communication interfaces 405 or indirectly by the indirect communication interfaces 406 via a connected external device(s) 407.

Here, the dotted line in FIG. 4 can also represent the optimal scanning distance 409 between the 3D body scanner 401 and the optimal scanning position 403.

The direction(s)/advice(s) given to the user may be of acoustical-, or optical or any other human noticeable nature. This can be, for example, an acoustic way like verbal (for example, voice) and/or non-verbal (for example, sound) and/or an optical way like written (static, running or animated text), static symbols or icons (for example, arrows or pictograms), dynamic (live) symbols, icons or animations.

The 3D body scanner 401 gives the user 412 some time to move.

These steps 1 to 7 of this interactive procedure are run in a loop until:

i. The user is found at step 5 to be within the circle of sufficient accuracy 408 and the 3D body scanner 401 gives the message that it is ready to scan or;
ii. The given maximum number of iterations is reached and the 3D body scanner 401 gives the message that a scan is not possible.

External device 407 can be, for example, but not limited to, a laptop, a smartphone, tablet and/or intelligent personal assistants.

An advantageous feature is that the user 412 is interactively guided to a scanning position in front of the 3D body scanner 401.

An advantageous feature is that the interaction between the 3D body scanner 401 and the user 412 is based on commands/directions given by the 3D body scanner 401 to the user 412 and the following reaction which is in the sense of the invention a change of body position which is noticed by the 3D body scanner 401 to create an ensuing command/direction.

An advantageous feature is that the position in front of the 3D body scanner 401 as far as possible optimally uses the field of view 413 of the 3D body scanner 401 with respect to installation height 103, 111 (see FIG. 1) and placement angle of the 3D body scanner 401 and the body height of the user 412 and potentially present obstacles.

An advantageous feature is that by intuitively accepting to follow the directions/advices of the 3D body scanner 401 to change the position of the body, no special user action is required.

An advantageous feature of the interactive instructions/advices is that there is no need for the user 412 to measure the distance and direction from the optimum scanning position 403 to the 3D body scanner 401 and/or to place a permanent or temporary marker for orientation on the floor.

An advantageous feature is that interactive guiding of the user 412 to the optimal scanning position 403 while the 3D body scanner 401 determines the geometry of installation and/or measures location data and then calculates by geometrical equations from the installation geometry and object locations, the optimum scanning position 403 and the actual position 402 of the user 412 on the floor 411, and from these the 3D body scanner 411 calculates the length and direction of the difference vector 404 between the actual position 402 of the user 412 and the optimum scanning position 403 and checks if the actual position of the user 402 is within a given circle of sufficient accuracy 408 close to the optimum scanning position 403 which ends the guiding process and the 3D body scanner 401 gives the message that it is ready to scan or alternatively, the 3D body scanner 401 gives the user a direction/advice how to move to the optimum scanning position 403 and then the 3D body scanner 401 gives the user 412 some time to move and finally restarts this process until the given maximum number of iterations is reached.

An advantageous feature is that the 3D body scanner 401 determines the geometry of installation and the location data of objects as, for example, but not limited to, walls, floor, persons or obstacles by sensing components of the 3D body scanner.

An advantageous feature is that for determination of the installation geometry, and optionally, if the user is already sufficiently visible, then also the body height of the user, the 3D body scanner 401 records, for example, but not limited to, depth map(s), with its depth sensor(s) and/or RGB or black-white image(s) by its camera(s) of the room in front of the 3D body scanner 401.

An advantageous feature is that for determination of the installation geometry, especially objects in front of the 3D body scanner 401, the 3D body scanner 401 measures in an advantageous way, location data as, for example, distance and direction of objects, as, for example, but not limited to, obstacles or persons or the user 412 by Lidar or microwave radar.

An advantageous feature is that the 3D body scanner 401 determines the optimum scanning position 403 and the actual position 402 of the user 412 and, if the user is already visible sufficiently, then also the body height of the user, by analyzing the installation geometry and/or, if available, location data.

An advantageous feature is that the interactive guiding is an iterative process terminated by the 3d body scanner upon the user reaching the circle of required accuracy 408 around the optimum scanning position 403 and/or that the given maximum number of iterations is reached.

An advantageous feature is that the direction/advice to the user 412 may be, for example, but not limited to, of acoustical-, or optical or any other human noticeable nature.

An advantageous feature is that the calculations for determining the optimum scanning position 403 and the actual position of the user 412 are, for example, but not limited to, done by a computation unit of the 3D body scanner 401, or within the computation unit of external device(s) 407 or somewhere in the cloud.

An advantageous feature is that the difference, shown as difference vector 404 between the optimum scanning position 403 and the actual position 402 of the user 412 (distance and/or direction) is used for guiding the user 412.

An advantageous feature is that the direction/advice to the user 412 may contain in advantageous way, for example, but not limited to, directional and/or distance information in absolute or relative scale and direction/advice can be given, for example, either by coordinates related to the main axis 409 of view of the 3D body scanner 401 or adaptively related to the direction 410 of view of the user 412.

An advantageous feature is that the directions/advices to the user 412 can be transmitted in an advantageous way, directly by the direct communication interfaces 405 and/or indirectly by the indirect communication interfaces 406 via a connected external device(s) 407.

An advantageous feature is that directions/advices to the user 412 can be provided, for example, but not limited to, in acoustic way as verbal (for example, natural or synthetic voice), non-verbal (for example, sound) and/or in an optical way as, for example, written (static, running or animated text), static symbols or icons (for example, arrows or pictograms), dynamic (live) symbols, icons or animations and/or any other human noticeable way.

### LIST OF REFERENCES

101: Floor
102: Low furniture
103: Height of low furniture
104: 3D body scanner (with control unit and optional accelerometer)
105: FOV of 3D body scanner
106: Person to be scanned at low placed scanner
107: Light or laser ray at low placed scanner
108: Angle of light or laser ray at low placed scanner
109: Optical indicator on floor at low placed scanner
110: High furniture
111: Height of high furniture
112: Person to be scanned at high placed scanner
113: Light or laser ray at low placed scanner
114: Angle of light or laser ray at high placed scanner
115: Optical indicator on floor at high placed scanner
116: Height axis of 3D body scanner
117: Starting distance of floor plane at low placed scanner
118: Starting distance of floor plane at high placed scanner
119: Viewed angle of floor plane at low placed scanner
120: Viewed angle of floor plane at high placed scanner
121: Optional RGB camera
201: Position indicator
202: Floor
203: Position indicator projector
204: Actuator
205: Light or laser ray
206: Tilt angle of light or laser ray
207: Control unit
209. Control signal for actuator
301: Position indicator projector
302: Electronic actuator (vector or line of light sources)
303: Active light source
304: Projection lens
305: Active light or laser ray
306: Optical indicator on floor
307: Floor
308: Farthest light indicator on floor
309: Closest light indicator on floor
310: Control unit
311: Angular (position) information
312: Control signals
313: Tilt angle of light or laser ray
314: Light sources
401: 3D body scanner
402: actual position of the user
403: optimal scanning position
404: difference vector
405: direct communication interface
406: indirect communication interface
407: external device
408: circle of sufficient position accuracy
409: main axis of view/optimal scanning distance
410: direction of view of the user
411: floor
412: user
413: field of view of the 3D body scanner

1. Method for operating a 3D body scanner for generating a 3D body model, which 3D body scanner is installed in an environment, which includes a floor, and ready for operation, the method comprising the following steps:
measuring an installation geometry of the 3D body scanner with respect to the environment,
using a control unit for calculating an optimal scanning distance between a person to be scanned and the 3D body scanner on basis of at least the installation geometry and
using a position indicator projector for projecting an optical indicator for the person to be scanned on the floor in front of the 3D body scanner at the optimal scanning distance from the 3D body scanner.

2. Method according to claim 1, wherein for the installation geometry an installation height of the 3D body scanner above the floor in front of the 3D body scanner is measured.

3. Method according to claim 2, wherein for calculating of the optimal scanning distance, the control unit takes into account a field of view of a depth sensor and a field of view of a color camera, of the 3D body scanner.

4. Method according to claim 3, further comprising the step of: scanning the field of view by the depth sensor and/or by the color camera and generating a depth map by the control unit on basis of the data of the depth sensor and/or of the color camera.

5. Method according to claim 4, further comprising the step of: identifying the floor in front of the 3D body scanner on basis of the depth map.

6. Method according to claim 4, wherein a body height of the person will be determined on basis of the depth map.

7. Method according to claim 4, further comprising the step of: calculating the height of the 3D body scanner above the floor and/or the angle of the 3D body scanner to the vertical axis on basis of the depth map.

8. Method according to claim 1, further comprising the step of: moving the optical indicator by controlling the position indicator projector.

9. Method according to claim 1, further comprising the step of: moving the optical indicator by controlling a two-dimensional matrix of light sources of an electronic actuator of the position indicator projector according to the optimal scanning position.

10. Method according to claim 1, further comprising the step of: guiding the person to be scanned to the optical indicator (109, 201, 306) by optical and/or audible signs.

11. A 3D body scanner for generating a 3D body model of a person to be scanned who is positioned in an environment, which includes a floor, in front of the 3D body scanner, the 3D body scanner comprising:
a depth sensor configured for measuring an installation geometry of the 3D body scanner with respect to the environment;
a control unit configured for calculating an optimal scanning distance between the person to be scanned and the 3D body scanner on basis of at least the installation geometry of the 3D body scanner; and
a position indicator projector configured for projecting an optical indicator at the optimal scanning distance on the floor of the environment.

12. A 3D body scanner according to claim 11, wherein the position indicator projector comprises a mirror and an actuator for tilting the mirror of the position indicator projector to deflect a light and/or a laser ray.

13. A 3D body scanner according to claim 11, wherein the position indicator projector comprises a light source selected from the group of light sources that includes LEDs, lasers, tungsten or halogen lamps;
wherein the position indicator projector comprises an electronic actuator with a two-dimensional matrix of light sources that includes a vector line of light sources, and
wherein the position indicator projector comprises a lens for projecting and/or focusing the light rays and/or the laser rays from the light source to the floor.

14. A 3D body scanner according to claim 11, wherein the 3D body scanner comprises an accelerometer for measuring the height of the 3D body scanner and/or an angle of the 3D body scanner according to a vertical axis.

15. A 3D body scanner according to claim 11, wherein the position indicator projector includes a plurality of light sources that emit light rays;
wherein the plurality of light sources of the position indicator projector are configured as a two-dimensional matrix of the plurality of light sources and includes a vector line of the plurality of light sources; and
wherein the position indicator projector includes a lens configured for projecting and/or focusing the light rays from the light source to the floor.

16. A 3D body scanner according to claim 11, wherein the position indicator projector includes a mirror and an actuator for tilting the mirror of the position indicator projector and wherein the position actuator includes a lens and is configured for adjusting the lens of the position indicator projector.

17. Method according to claim 1, wherein an installation angle of the 3D body scanner relative to a vertical axis is determined during the step of measuring the installation geometry of the 3D body scanner with respect to the floor.

18. Method according to claim 1, wherein during the step of calculating of the optimal scanning distance, the control unit takes into account a field of view of a depth sensor and a field of view of a RGB camera of the 3D body scanner.

19. Method according to claim 18, further comprising the step of: using the control unit for scanning the field of view by the depth sensor and/or by the RGB camera and generating a depth map on basis of the data of the depth sensor and/or of the RGB camera.

20. Method according to claim 19, further comprising the step of: calculating the height of the 3D body scanner above the floor and/or the angle of the 3D body scanner to a vertical axis on basis of the depth map.
